(19) United States
(12) Patent Application Publication
(10) Pub. No.: US 2019/0364946 A1
(43) Pub. Date: Dec. 5, 2019

(54) POWDERS AND TABLETS COMPRISING OMEGA-3 FATTY ACID DERIVATIVES AND METHODS FOR THEIR PRODUCTION

(71) Applicant: OMEGATRI AS, Oslo (NO)

(72) Inventors: Tina LIEN VESTLAND, Oslo (NO); Jo KLAVENESS, Oslo (NO)

(21) Appl. No.: 16/085,178

(22) PCT Filed: Mar. 15, 2017
(86) PCT No.: PCT/IB2017/000548

§ 371 Date: Sep. 14, 2018

Related U.S. Application Data
(60) Provisional application No. 62/309,013, filed on Mar. 16, 2016.

(51) Int. Cl. A23L 29/00; A23L 33/12; A61K 31/202; A61K 9/28; A23P 10/22; A23P 10/40; A23P 10/28

(52) U.S. Cl. CPC A23L 29/04; A23L 33/12; A61K 31/202; A23P 10/28; A61K 9/2866; A23P 10/22; A23P 10/40; A61K 9/2846

(57) ABSTRACT

The present invention relates to methods of using surface active compounds(s) in the preparation process for powders comprising beta-cyclodextrin and omega-3 fatty acids and derivatives thereof and to the dry powders and tablets comprising surface active compounds(s) preferably diglycerides, beta-cyclodextrin and omega-3 fatty acids and derivatives thereof.

### FIELD OF THE INVENTION

The present invention relates to an improved method for preparation of powder and tablets comprising omega-3 fatty acid derivatives and beta-cyclodextrin.

### BACKGROUND OF THE INVENTION

Omega-3 comprising products are generally provided either in the form of oil encapsulated in soft capsules or in the form of free oil (cod liver oil products). There has been a need for omega-3 products with different and improved properties relative to omega-3 in the form of oil. These improved properties are one or more of the following: improved oxidative stability, reduced fish taste typically from gastrointestinal reflux, efficient uptake from the gastrointestinal system, improved technical possibilities to prepare stable combination products (products comprising omega-3 plus one or more active components like minerals, vitamins, drug substances or food additives) and finally omega-3 products that can be used in food products like drinks (e.g. juice) and semisolid/solid food products (e.g. yoghurt and bread). Various forms of dry powders based on encapsulation of omega-3 droplets have been developed and these dry powders are extensively used in various drug products. These powders based on physical encapsulation of omega-3 oil do not have all the properties listed above and cannot be tableted due to the high pressure and increased temperature during tableting.

### SUMMARY OF THE INVENTION

The present invention relates to an improved method for preparation of powder and tablets comprising omega-3 fatty acid derivatives and beta-cyclodextrin.

Accordingly, in some embodiments, the present invention provides compositions comprising a dry powder comprising beta-cyclodextrin in an amount of from 60% to 90% w/w of the powder and a lipid component in an amount of from about 10% to 40% w/w of the powder, wherein the lipid component is characterized as having a surfactant content of from about 0.1% to 35% w/w of the lipid component.

In some embodiments, the lipid component comprises an omega-3 fatty acid or derivative thereof selected from the group consisting of omega-3 triglycerides, omega-3 ethyl esters, omega-3 free fatty acids and salts of omega-3 fatty acids. In some embodiments, wherein the omega-3 fatty acids or derivatives thereof have an EPA:DHA ratio of greater than 1:1. In some embodiments, the omega-3 fatty acids or derivatives thereof have a DHA:EPA ratio of greater than 1:1. In some embodiments, the omega-3 triglycerides are a marine oil. In some embodiments, the marine oil is selected from the group consisting of fish oil, squid oil and algal oil. In some embodiments, the omega-3 fatty acid or derivative thereof selected from the group consisting of omega-3 triglycerides, omega-3 ethyl esters, omega-3 free fatty acids and salts of omega-3 fatty acids comprises EPA and DHA fatty acids at a concentration of from 10% to 99% w/w of the fatty acids in the omega-3 triglycerides, omega-3 ethyl esters, omega-3 free fatty acids and salts of omega-3 fatty acids. In some embodiments, the omega-3 fatty acid or derivative thereof selected from the group consisting of omega-3 triglycerides, omega-3 ethyl esters, omega-3 free fatty acids and salts of omega-3 fatty acids comprises EPA and DHA fatty acids at a concentration of from 10% to 70% w/w of the fatty acids in the omega-3 triglycerides, omega-3 ethyl esters, omega-3 free fatty acids and salts of omega-3 fatty acids. In some embodiments, the omega-3 fatty acid or derivative thereof selected from the group consisting of omega-3 triglycerides, omega-3 ethyl esters, omega-3 free fatty acids and salts of omega-3 fatty acids comprises EPA and DHA fatty acids at a concentration of from 30% to 60% w/w of the fatty acids in the omega-3 triglycerides, omega-3 ethyl esters, omega-3 free fatty acids and salts of omega-3 fatty acids.

In some embodiments, the surfactant is selected from the group consisting of mono- and diglycerides of fatty acids, sorbitan esters of fatty acids, and polysorbates and combinations thereof. In some embodiments, the surfactant is selected from the group consisting of mono- and diglycerides of fatty acids and combinations thereof. In some embodiments, the surfactant is a diglyceride of fatty acids. In some embodiments, the diglycerides of fatty acids comprise a mixture of diglyceride compounds wherein the fatty acid components of the diglycerides compounds are selected from saturated, monounsaturated and polyunsaturated fatty acids. In some embodiments, the polyunsaturated fatty acids are omega-3 fatty acids. In some embodiments, the omega-3 fatty acids are selected from EPA and DHA. In some embodiments, the concentration of the surfactant in the lipid component is from 10% to 35% w/w of the lipid component. In some embodiments, the surfactant is not an added naturally occurring surfactant selected from the group consisting of naturally occurring phospholipids, triglycerides and free fatty acids or a salt or ester of a long chain omega-3 fatty acid.

In some embodiments, the powder composition is spray granulated. In some embodiments, the powder composition is spray granulated and has a particle size distribution of 50-650 microns. In some embodiments, the powder composition is spray granulated and has a particle size distribution of 200-500 microns.

In some embodiments, the present invention provides tableted lipid formulations comprising beta-cyclodextrin in a concentration of from 60% to 90% w/w of the tablet and a lipid component in a concentration of from 10% to 40% w/w of the tablet, wherein the lipid component is characterized as having a surfactant content of from 0.1% to 35% w/w of the lipid component, wherein the tablet has a crushing strength of greater than 3 kN.

In some embodiments, the tablet has a crushing strength of greater than 5 kN. In some embodiments, the tablet has a crushing strength of greater than 7 kN. In some embodiments, the tablet has a crushing strength of from 5 to 10 kN.

In some embodiments, the present invention provides tableted lipid formulations comprising beta-cyclodextrin in a concentration of from 60% to 90% w/w of the tablet and a lipid component in a concentration of from 10% to 40% w/w of the tablet, wherein the lipid component is characterized as having a diglyceride content of from 10% to 35% w/w of the lipid component, wherein the tablet has a crushing strength of greater than 3 kN.

In some embodiments, the tablet has a crushing strength of greater than 5 kN. In some embodiments, the tablet has a crushing strength of greater than 7 kN. In some embodiments, the tablet has a crushing strength of from 5 to 10 kN.

In some embodiments, the lipid component comprises an omega-3 fatty acid or derivative thereof selected from the group consisting of omega-3 triglycerides, omega-3 ethyl esters, omega-3 free fatty acids and salts of omega-3 fatty acids. In some embodiments, the omega-3 fatty acids or derivatives thereof have an EPA:DHA ratio of greater than 1:1. In some embodiments, the omega-3 fatty acids or derivatives thereof have a DHA:EPA ratio of greater than 1:1. In some embodiments, the omega-3 triglycerides are a marine oil. In some embodiments, the marine oil is selected from the group consisting of fish oil, squid oil and algal oil. In some embodiments, the omega-3 fatty acid or derivative thereof selected from the group consisting of omega-3 triglycerides, omega-3 ethyl esters, omega-3 free fatty acids and salts of omega-3 fatty acids comprises EPA and DHA fatty acids at a concentration of from 10% to 99% w/w of the fatty acids in the omega-3 triglycerides, omega-3 ethyl esters, omega-3 free fatty acids and salts of omega-3 fatty acids. In some embodiments, the omega-3 fatty acid or derivative thereof selected from the group consisting of omega-3 triglycerides, omega-3 ethyl esters, omega-3 free fatty acids and salts of omega-3 fatty acids comprises EPA and DHA fatty acids at a concentration of from 10% to 70% w/w of the fatty acids in the omega-3 triglycerides, omega-3 ethyl esters, omega-3 free fatty acids and salts of omega-3 fatty acids. In some embodiments, the omega-3 fatty acid or derivative thereof selected from the group consisting of omega-3 triglycerides, omega-3 ethyl esters, omega-3 free fatty acids and salts of omega-3 fatty acids comprises EPA and DHA fatty acids at a concentration of from 30% to 60% w/w of the fatty acids in the omega-3 triglycerides, omega-3 ethyl esters, omega-3 free fatty acids and salts of omega-3 fatty acids.

In some embodiments, the diglycerides comprise a mixture of diglyceride compounds wherein the fatty acid components of the diglycerides compounds are selected from saturated, monounsaturated and polyunsaturated fatty acids. In some embodiments, the polyunsaturated fatty acids are omega-3 fatty acids. In some embodiments, omega-3 fatty acids are selected from EPA and DHA. In some embodiments, the tableted lipid formulation does not comprise an added naturally occurring surfactant selected from the group consisting of naturally occurring phospholipids, triglycerides and free fatty acids or a salt or ester of a long chain omega-3 fatty acid.

In some embodiments, the tableted formulation is coated. In some embodiments, the tableted formulation is coated with an agent selected from the group consisting of polyvinyl acetate, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, cellulose acetate trimellitate, and sodium alginate.

In some embodiments, the lipid component is combined with an additional nutraceutical agent that is not an omega-3 fatty acid or derivative thereof. In some embodiments, the lipid component is combined with an additional pharmaceutical agent that is not an omega-3 fatty acid or derivative thereof.

In some embodiments, the present invention provides processes for making a tabletable lipid powder comprising: combining an aqueous solution of beta-cyclodextrin with a lipid component in an amount of from 10% to 40% w/w of the beta-cyclodextrin the solution, wherein the lipid component comprises one or more surfactants at a concentration of from 0.1% to 35% w/w of the lipid component; mixing the aqueous solution of beta-cyclodextrin and the lipid component to provide a mixture; and removing water from the mixture to provide a dry powder.

In some embodiments, the lipid component comprises an omega-3 fatty acid or derivative thereof selected from the group consisting of omega-3 triglycerides, omega-3 ethyl esters, omega-3 free fatty acids and salts of omega-3 fatty acids. In some embodiments, the omega-3 fatty acids or derivatives thereof have an EPA:DHA ratio of greater than 1:1. In some embodiments, the omega-3 fatty acids or derivatives thereof have a DHA:EPA ratio of greater than 1:1. In some embodiments, the omega-3 triglycerides are a marine oil. In some embodiments, the marine oil is selected from the group consisting of fish oil, squid oil and algal oil. In some embodiments, the omega-3 fatty acid or derivative thereof selected from the group consisting of omega-3 triglycerides, omega-3 ethyl esters, omega-3 free fatty acids and salts of omega-3 fatty acids comprises EPA and DHA fatty acids at a concentration of from 10% to 99% w/w of the fatty acids in the omega-3 triglycerides, omega-3 ethyl esters, omega-3 free fatty acids and salts of omega-3 fatty acids. In some embodiments, the omega-3 fatty acid or derivative thereof selected from the group consisting of omega-3 triglycerides, omega-3 ethyl esters, omega-3 free fatty acids and salts of omega-3 fatty acids comprises EPA and DHA fatty acids at a concentration of from 10% to 70% w/w of the fatty acids in the omega-3 triglycerides, omega-3 ethyl esters, omega-3 free fatty acids and salts of omega-3 fatty acids. In some embodiments, the omega-3 fatty acid or derivative thereof selected from the group consisting of omega-3 triglycerides, omega-3 ethyl esters, omega-3 free fatty acids and salts of omega-3 fatty acids comprises EPA and DHA fatty acids at a concentration of from 20% to 45% w/w of the fatty acids in the omega-3 triglycerides, omega-3 ethyl esters, omega-3 free fatty acids and salts of omega-3 fatty acids.

In some embodiments, the surfactant is selected from the group consisting of selected among mono- and diglycerides of fatty acids, sorbitan esters of fatty acids, and polysorbates and combinations thereof. In some embodiments, the surfactant is selected from the group consisting of mono- and diglycerides of fatty acids and combinations thereof. In some embodiments, the surfactant is a diglyceride of fatty acids. In some embodiments, the diglycerides of fatty acids comprise a mixture of diglyceride compounds wherein the fatty acid components of the diglycerides compounds are selected from saturated, monounsaturated and polyunsaturated fatty acids. In some embodiments, the polyunsaturated fatty acids are omega-3 fatty acids. In some embodiments, the omega-3 fatty acids are selected from EPA and DHA. In some embodiments, the concentration of the surfactant in the lipid component is from 10% to 35% w/w of the lipid component. In some embodiments, the surfactant is not an added naturally occurring surfactant selected from the group consisting of naturally occurring phospholipids, triglycerides and free fatty acids or a salt or ester of a long chain omega-3 fatty acid.

In some embodiments, the removing water from the mixture to provide a dry powder further comprises spray drying. In some embodiments, the removal of water is performed as spray granulation and the powder has a particle size distribution of 50-650 microns. In some embodiments, the removal of water is performed as spray granulation and the powder has a particle size distribution of 200-500 microns.

In some embodiments, the processes further comprise the step of forming a tablet from the dry powder. In some embodiments, the tablet has a crushing strength of greater than 5 kN. In some embodiments, the tablet has a crushing strength of greater than 7 kN. In some embodiments, the tablet has a crushing strength of from 5 to 10 kN.

### DETAILED DESCRIPTION OF THE INVENTION

The present invention relates to the method to use surface active compounds(s), preferably diglycerides, in the preparation process for powders comprising beta-cyclodextrin and omega-3 fatty acids and derivatives thereof and to the dry powders and tablets comprising surface active compounds(s), preferably diglycerides, beta-cyclodextrin and omega-3 fatty acids and derivatives thereof.

The inventors unexpectedly observed that the addition of between 10% and 35% diglycerides to an omega-3 oil composition (% weight/weight (w/w) calculated as weight of diglycerides divided by the total weight of the diglycerides plus the weight of the oil) allowed for the production of a beta-cyclodextrin powder with superior tabletting properties as well as tablets with superior properties.

The most preferred diglycerides for use in the present invention comprise a mixture of diglyceride compounds, where the fatty acid components in the mixture of diglyceride molecules can be saturated, monounsaturated and/or polyunsaturated, including omega-3 fatty acids like EPA and DHA.

The diglyceride molecules typically comprise saturated, monounsaturated and/or polyunsaturated fatty acids of various number of carbon atoms and various number of double bonds. Some typical fatty acids include fatty acids belonging to one or more of the following groups of fatty acids: 14:0, 15:0, 16:0, 16: 1, 17:0, 18:0, 18: 1, 18:2, 18 :3, 18: 4, 20: 1, 20: 4, 20:5, 22: 1, 22:5 and 22:6. The first number represents the number of carbon atoms and the last number represents the number of double bonds. EPA belongs to the fatty acid group 20:5 with 20 carbon atoms and 5 double bonds, while DHA belongs to the fatty acid group 22:6 with 22 carbon atoms and 6 double bonds. Both EPA and DHA have cis (Z)-isomer double bonds.

The diglycerides described according to the present invention might be naturally present in the oil or present in the oil as a result of the production process. The diglyceride might also be added to the oil before processing to a tablettable powder. In any event, the diglyceride content of the oil is adjusted to be in the range of from 10% to 35% of the weight of the oil on a w/w basis.

The diglyceride can be in the form of 1,2-diacylglycerols and/or 1,3-diacylglycerols. Diglycerides of fatty acids are approved food additives as emulsifiers. The diglycerides preferably have an HLB of about 3.

One of the most preferred aspects of the present invention is to use oil comprising a mixture of diglycerides where one or more of the single diglyceride molecular components are diglycerides with one or two EPA fatty acids or diglycerides with one or two DHA fatty acids.

If one component in the diglyceride mixture is a diglyceride with EPA, the other fatty acid might typically be an acid selected among the following groups of fatty acids: 14:0, 15:0, 16:0, 16: 1, 17:0, 18:0, 18: 1, 18:2, 18 :3, 18: 4, 20: 1, 20: 4, 20:5, 22: 1, 22:5 and 22:6.

If one component in the diglyceride mixture is a diglyceride with DHA, the other fatty acid might typically be an acid selected among the following groups of fatty acids: 14:0, 15:0, 16:0, 16: 1, 17:0, 18:0, 18: 1, 18:2, 18 :3, 18: 4, 20: 1, 20: 4, 20:5, 22: 1, 22:5 and 22:6.

The inclusion of such surfactant(s), preferably diglycerides, improve(s) the quality of the powder both with regard to stability and tabletability. The present invention therefor relates to methods for preparation of powder and tablets comprising omega-3 fatty acid derivatives and beta-cyclodextrin and products prepared by the method. The method is characterized by including of one or more surface active compounds preferably diglycerides during preparation of the aqueous slurry before preparation of the dry powder. The surfactant will generally be present in the powder and thereby the tablets. Aspects of the present invention are therefore powders and tablets comprising omega-3 fatty acid derivatives, beta-cyclodextrin and one or more surface active compounds. In some preferred embodiments, the surface active agent is not a phospholipid and in further preferred embodiments, the powders contain less than 10%, 5%, 1% or 0.1% w/w total phospholipids. The method to use surface active compounds(s) in the preparation process for powders comprising beta-cyclodextrin and omega-3 is new and the obtained powders showed unexpected improved properties. Dry powders and tablets comprising omega-3 fatty acids and derivatives thereof, beta-cyclodextrin, and surface active compound(s) preferably diglycerides are also new.

The description below describes the following aspects of the present invention: (1) Method; (2) Powder prepared according to the method; and (3) Tablets comprising omega-3, cyclodextrin and surfactants, preferably diglycerides.

### 1. Method

One aspect of the present invention relates to a method for preparation of dry powder comprising omega-3, beta-cyclodextrin using a surfactant preferably diglycerides. A preferred aspect of the method is a method for preparation of dry powder comprising omega-3, beta-cyclodextrin using a surfactant, preferably diglycerides, where the dry powder is a tabletable powder. A more preferred aspect of this aspect of method is a method for preparation of dry powder comprising omega-3, beta-cyclodextrin using a surfactant, preferably diglycerides, where the dry powder is a tabletable powder that can be tableted using standard tableting equipment producing more than 10,000 tablets per hour and the tablets can be prepared continuously for hours.

In preferred embodiments, beta-cyclodextrin, one or more omega-3 fatty acid and derivatives thereof, and a surfactant, preferably diglycerides, are combined in an aqueous mixture. The mixture is agitated, for example by stirring, for a period of from about 5 minutes to 300 minutes, preferably from about 30 to about 90 minutes, and most preferably for about 60 minutes. The water is then removed from the mixture, for example by evaporation under reduced pressure to yield a dry, tabletable powder. In some embodiments, the water is preferably removed by spray drying or granulation. In some embodiments, the powder has a particle size distribution of 50-650 microns, and most preferably from 200-500 microns, following spray drying or granulation.

In some embodiments, from 5% to 40%, 10% to 40%, 20% to 40%, 30% to 40%, 5% to 35%, 10% to 35%, 20% to 35%, 25% to 35%, 30% to 35%, at least 5%, at least 10%, at least 20%, at 30% or at least 35% w/w of an oil component is combined with the beta-cyclodextrin, wherein w/w refers to the total weight of the oil component to the total weight of beta-cyclodextrin. The w/w % above includes the weight of surfactant (e.g., a diglycerides) in the oil used according to the present invention. In some embodiments, the oil component preferably comprises one or more omega-3 fatty acids or derivatives thereof. In some embodiments, the one or more omega-3 fatty acids or derivatives thereof are selected from omega-3 triglycerides, omega-3 ethyl esters, free omega-3 acids and/or pharmaceutically acceptable or food acceptable quality omega-3 fatty acid salts, alone or in combination. In some embodiments, one or more of these omega 3 fatty acids and derivatives thereof are the main omega-3 components used to prepare the dry powder. In some embodiments, one or more of these omega 3 fatty acids and derivatives thereof are the main oil components used to prepare the dry powder (e.g., the oil component used in the process comprises greater than about 60%, 70%, 80%, 90%, or 95% w/w of the specified omega-3 fatty acid or derivative thereof (omega-3 triglycerides, omega-3 ethyl esters, free omega-3 acids and/or pharmaceutically acceptable or food acceptable quality omega-3 fatty acid salts) wherein w/w refers to the total weight of the specified omega-3 fatty acid or derivative thereof per the total weight of the oil component). In some embodiments, one or more of these omega 3 fatty acids and derivatives thereof are from a marine source, such as fish, algae, or have been prepared from raw products from fish or algae. In some embodiments, one or more of these omega 3 fatty acids and derivatives thereof are from plants or vegetables or have been prepared from raw products from plants and vegetables.

In some embodiments, the lipid component used to prepare the dry powder is an omega-3 composition. In some embodiments, the dry powder is prepared with omega-3 triglycerides. In some embodiments, the dry powder is prepared with omega-3 ethyl esters.

In some embodiments, the dry powder is prepared with free omega-3 acids. In some embodiments, the dry powder is prepared with pharmaceutically acceptable or food acceptable quality of omega-3 fatty acid salts. In some embodiments, the omega-3 fatty acids or derivatives are preferably selected from EPA and DHA and combinations thereof. In some embodiments, the omega 3 fatty acids and derivatives thereof comprise more EPA than DHA. In some embodiments, the omega 3 fatty acids and derivatives thereof comprise more DHA than EPA. In some embodiments, the omega 3 fatty acids and derivatives thereof (e.g., triglycerides, ethyl esters, free acids or salts thereof, alone or in combination) comprise are enriched for EPA, e.g., more than 90% w/w of the total omega 3 fatty acids and derivatives thereof in the powder is EPA where the w/w % is the weight of EPA per total weight of fatty acids in the powder. In some embodiments, the omega 3 fatty acids and derivatives thereof (e.g., triglycerides, ethyl esters, or free acids) are enriched for DHA, e.g., more than 90% w/w of the total omega 3 fatty acids and derivatives thereof in the powder is DHA where the w/w % is the weight of DHA per total weight of fatty acids in the powder. In some embodiments, the lipid component preferably comprises from about 30% to 60% w/w EPA and/or DHA.

In some preferred embodiments, from 0.1% to 10% w/w, 0.1% to 20% w/w, 0.1% to 30% w/w, 1% to 10% w/w, 1% to 20% w/w, 1% to 30% w/w, 2% to 10% w/w, 2% to 20% w/w, 2% to 30% w/w, 5% to 10% w/w, 5% to 20% w/w, 5% to 30% w/w, 10% to 20% w/w, 10% to 30% w/w, 15% to 30% w/w, 18% to 30% w/w, 20% to 30% w/w surfactant or combination of surfactants is included with the oil component, wherein w/w refers to the total weight of surfactant (or combination thereof) to the total weight of the oil component. In some embodiments, the surfactant is a surfactant approved for use for preparation of pharmaceutical products and or approved for use in food products. In some embodiments, the surfactant is a surfactant approved for use for preparation of pharmaceutical products. In some embodiments, the surfactant is a surfactant approved for use for preparation of approved for use in food products. In some embodiments, the surfactant is a ionic surfactant; preferably a negatively charged surfactant. In some embodiments, the surfactant is a non-ionic surfactant. In some embodiments, the surfactant is a naturally occurring surfactant. In some embodiments, the surfactant is a surfactant produced synthetically or party produced synthetically. In some embodiments, the surfactant is a derivative of a fatty acid. In some embodiments, the surfactant is a derivative of glycerol. In some embodiments, the surfactant is selected among substances that are permitted to be used as food additives for use within the European Union, US or Asia. In some embodiments, the surfactant is selected among substances listed on the GRAS list. In some embodiments, the surfactant is selected among the following compounds: E 400 alginic acid, E401 sodium alginate, E402 potassium alginate, E403 ammonium alginate, E404 calcium alginate, E430 polyoxyethene (8) stearate, E431 polyoxyethene (40) stearate, E432 polyoxyethene (20) sorbitan monolaurate (polysorbate 20), E433 polyoxyethene (20) sorbitan monooleate (polysorbate 80), E434 polyoxyethene (20) sorbitan monopalmitate (polysorbate 40), E435 polyoxyethene (20) sorbitan monostearate (polysorbate 60), E436 polyoxyethene (20) sorbitan tristearate (polysorbate 65), E470a sodium, potassium and calcium salts of fatty acids, E470b magnesium salts of fatty acids, E471 mono- and diglycerides of fatty acids (glyceryl monostearate, glyceryl distearate) and other monoglycerides of fatty acids and diglycerides of fatty acids, E472 acetic acid esters of mono- and diglycerides of fatty acids, E472b lactic acid esters of mono- and diglycerides of fatty acids, E472c citric acid esters of mono- and diglycerides of fatty acids, E472d tartaric acid esters of mono- and diglycerides of fatty acids, E472e mono- and diacetyl tartaric acid esters of mono- and diglycerides of fatty acids, E472f mixed acetic and tartaric acid esters of mono- and diglycerides of fatty acids, E472g succinylated monoglycerides, E473 sucrose esters of fatty acids, E474 sucroglycerides, E475 polyglycerol esters of fatty acids, E476 polyglycerol polyricinoleate, E477propane-1,2-diol esters of fatty acids, propylene glycol esters of fatty acids, E478 lactylated fatty acid esters of glycerol and propane-1, E479b thermally oxidized soya bean oil interacted with mono- and diglycerides of fatty acid, E480 dioctyl sodium sulphosuccinate, E481 sodium stearoyl-2-lactylate, E482 calcium stearoyl-2-lactylate, E483 stearyl tartrate, E484 stearyl citrate, E485 sodium stearoyl fumarate, E486 calcium stearoyl fumarate, E487 sodium laurylsulphate, E488 ethoxylated mono- and di-glycerides, E489 methyl glucoside-coconut oil ester, E490 propane-1,2-diol, E491 sorbitan monostearate, E492 sorbitan tristearate, E493 sorbitan monolaurate, E494 sorbitan monooleate, E495 sorbitan monopalmitate, E496 sorbitan trioleate, E497 polyoxypropylene-polyoxyethylene polymers and E498 partial polyglycerol esters of polycondensed fatty acids of castor oil. The term fatty acid includes any natural saturated fatty acids, monounsaturated fatty acids and polyunstaturated fatty acids and mixtures thereof.

In some embodiments, the surfactant or a surfactant mixture that has a HLB value of from 1 to 20. The HLB (Hydrophilic Lipophilic Balance) value for a given surfactant is measure of the degree to which the surfactant is hydrophilic or lipophilic. The figure is dependent on which functional groups that are present in the surfactant molecule and where in the molecule these functional groups are located. Surfactants with HLB value of less than 10 are soluble in lipids, while surfactants with HLB values higher than 10 are soluble in water. The HLB values of the various surfactants are available from various commercial and scientific sources; see for example Surfactants Classified by HLB Numbers on sigmaaldrich.com or basic teaching books in pharmaceutical sciences like A. T. Florence and D. Attwood: Physicochemical Principles of Pharmacy, Pharmaceutical Press, 2004 on page 240. The HLB value for some preferred surfactants according to the present invention are: mono-and diglycerides (HLB=appr.2-5 (depending on ratio, the more diglyceride the lower HLB value)), sorbitan esters HLB values around 4-5 (sorbitan oleate HLB=4.3, sorbitan monostearate HLB=4.7, sorbitan stearate HLB=4.7) and polysorbates HLB values around 15.

In some preferred embodiments, the surfactant is selected among mono- and diglycerides of fatty acids, sorbitan esters with fatty acids and polysorbates or mixtures thereof. In some embodiments, the surfactant is not a phospholipid and most preferably is not a naturally occurring phospholipid. In some embodiments, the surfactant is not a triglyceride and most preferably is not a naturally occurring triglyceride. In some embodiments, the surfactant is not a free fatty acid and most preferably is not a naturally occurring free fatty acid. In some preferred embodiments, the surfactant is not a salt or ester of EPA, DHA or other long chain (greater than 20 carbons) omega-3 fatty acid.

The more preferred surfactants are diglycerides and the most preferred surfactants are diglycerides where one or more of the acids are omega-3 fatty acids. In especially preferred embodiments, the diglycerides comprise of a mixture of diglyceride compounds where the fatty acid components in the mixture of diglyceride molecules can be saturated, monounsaturated and polyunsaturated including omega-3 fatty acids like EPA and DHA.

A person skilled in the art would expect based on the unexpectedly good results with omega-3 oils comprising diglyceride for preparation of tablettable powder and tablets that other surfactants or surfactant mixtures with similar HLB-values will be as helpful as diglycerides.

In some embodiments, the lipid component preferably comprises an omega-3 fatty acid or derivative thereof selected from the group consisting of omega-3 triglycerides, omega-3 ethyl esters, omega-3 free fatty acids and salts of omega-3 fatty acids. However, in some embodiments, additional active ingredients may be included in the lipid component along with omega-3 fatty acids or derivatives thereof. Suitable additional active ingredients include, but are not limited to other active fatty acids such as omega-6 fatty acids, conjugated fatty acids such as conjugated linoleic acid fatty acid, and lipophilic drugs such as as Class II and Class IV drugs as classified under the Biopharmaceutics Classification System. Indeed, a variety a nutraceutical and pharmaceutical agents may be included in the lipid component. In some preferred embodiments, the nutraceutical and pharmaceutical agents are lipophilic.

In some embodiments, the active ingredient is a pharmaceutical ingredient selected from the groups consisting of antineoplastic, antifungal, antiviral, anticonvulsant, antiepileptic, immunosuppressant, and erectile dysfunction drugs. The BCS is a guide for predicting the intestinal drug absorption provided by the U.S. Food and Drug Administration. This system restricts the prediction using the parameters solubility and intestinal permeability. According to the Biopharmaceutics Classification System, drug substances are classified as follows: Class I—high permeability, high solubility (compounds are well absorbed at all GI PH and their absorption rate is usually higher than excretion); Class II—high permeability, low solubility (bioavailability of those products is limited by their solubility and rate of dissolution Class III—low permeability, high solubility (absorption is limited by the permeation rate but the drug is solvated very fast; if the formulation does not change the permeability or gastro-intestinal duration time, then class I criteria can be applied); Class IV—low permeability, low solubility (compounds have a poor bioavailability; usually they are not well absorbed over the intestinal mucosa and a high variability is expected).

The drugs are classified in BCS on the basis of following parameters: 1. Solubility; 2.Permeability; and 3. Rate of dissolution. Solubility class boundaries are based on the highest dose strength of an immediate release product. A drug is considered highly soluble when the highest dose strength is soluble in 250m1 or less of aqueous media over the ph range of Ito 7.5. The volume estimate of 250m1 is derived from typical bioequivalence study protocols that prescribe administration of a drug product to fasting human volunteers with a glass of water. Permeability class boundaries are based indirectly on the extent of absorption of a drug substance in humans and directly on the measurement of rates of mass transfer across human intestinal membrane. Alternatively non-human systems capable of prediction the drug absorption systems capable of predicting the drug absorption in humans can be used (such as in-vitro culture methods). A drug substance is considered highly permeable when the extent of absorption in humans is determined to be 90% or more of the administered dose based on a mass-balance determination or in comparison to and intravenous dose. With respect to dissolution class boundaries, an immediate release products is considered rapidly dissolving when no less than 85% ofthe labeled amount of the drug substance dissolve within 15 minutes using USP Dissolution Apparatus 1 at 100 RPM or Apparatus 2 at 50 RPM in a volume of 900 ml or less in following media,) 0.1 N HCl or simulated gastric fluid or pH 4.5 buffer and pH 6.8 buffer or simulated intestinal fluid.

In some embodiments, the additional active ingredient is a drug including, but not limited to the following drugs: tripranavir, cefditoren pivoxil, tadalafil, mycophenolic acid, posaconazole, lapatinib, bromocriptine, ticagrelor, sorafetinib, itraconazole, erlotinib, sirolimus, alvimopan, naltrexone, vardenafil, rosuvastatin, maraviroc, ritonavir, efavirez, celecoxib, atovaquone, raloxifene, finasteride, everolimus, and dodrenarone.

In some embodiments, the additional active ingredient is selected from the groups consisting of antineoplastic, antifungal, antiviral, anticonvulsant, antiepileptic, antidepressant, immunosuppressant, anti-inflammatory and erectile dysfunction drugs.

In some embodiments, exemplary antineoplastic drugs suitable for use as an additional active ingredient include, but are not limited to: I) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPTII), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; and 6) platinum compounds (e.g., cisplatin and carboplatin, etc.).

In some embodiments, exemplary antifungal drugs suitable for use as an additional active ingredient include, but are not limited to nystatin, amphotericin B, griseofulvin, miconazole, ketoconazole, terbinafine, itraconazole, fluconazole, posaconazole, and voriconazole. In some embodiments, exemplary antiviral drugs suitable for use in dosage forms of the present invention include, but are not limited to abacavir, aciclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, lamivudine, lopinavir, loviride, maraviroc, moroxydine, methisazone, nelfinavir, nevirapine, nexavir, oseltamivir (Tamiflu), peginterferon alfa-2a, penciclovir, peramivir, pleconaril, podophyllotoxin, raltegravir, ribavirin, rimantadine, ritonavir, pyramidine, saquinavir, stavudine, tea tree oil, tenofovir, tenofovir disoproxil, tipranavir, trifluridine, trizivir, tromantadine, truvada, valaciclovir (Valtrex), valganciclovir, vicriviroc, vidarabine, viramidine, zalcitabine, zanamivir (Relenza) and zidovudine.

In some embodiments, exemplary anticonvulsant drugs suitable for use as an additional active ingredient include, but are not limited to pregabalin, gabapentin, carbamazepine, and oxcarbazepine.

In some embodiments, exemplary antiepileptic and anticonvulsant drugs suitable for use as an additional active ingredient include, but are not limited to pregabalin, gabapentin, carbamazepine, and oxcarbazepine and alprazolam, bretazenil, bromazepam, brotizolam, chlordiazepoxide, cinolazepam, clonazepam, clorazepate, clotiazepam, cloxazolam, delorazepam, diazepam, estazolam, etizolam, flunitrazepam, flurazapam, flutoprazepam, halazepam, ketazolam, loprazolam, lorazepam, lormetazepam, medazepam, midazolam, nemetazepam, nitrazepam, nordazepam, oxazepam, phenazepam, pinazepaam, prazepam, premazepam, quazepam, temazepam, tetrazepam, triazolam, clobazam, DMCM, flumazenil, eszopiclone, zaleplon, zolpidem, and zopiclone.

In some embodiments, exemplary antidepressant drugs suitable for use as an additional active ingredient include, but are not limited to tricyclic compounds such as bupropion, nortriptyline, desipramine, amitriptyline, amitriptylinoxide, butriptyline, clomipramine, demexiptiline, dibenzepin, dimetacrine, dosulepin/dothiepin, doxepin, imipramine, amineptine, iprindole, opipramol, tianeptine, trimipramine, imipraminoxide, lofepramine, melitracin, metapramine, nitroxazepine, noxiptiline, pipofezine, propizepine, protriptyine, and quinupramine; SNRis such as duloxetine, venlafaxine, desvenlafaxine, milnacipran, levomilnacipran, sibutramine, bicifadine, and SEP-227162; and SSRis such as citalopram, dapoxetine, escitalopram, fluoxetine, fluvoxamine, indalpin, paroxetine, sertraline, and zimelidine.

In some embodiments, exemplary immunosuppressant drugs suitable for use as an additional active ingredient include, but are not limited to azathioprine, mycophenolic acid, leflunomide, teriflunomide, methotrexate, tacrolimus, cyclosporin, pimecrolimus, abetimus, gusperimus, thalidomide, lenalidomide, anakinra, sirolimus, everolimus, ridaforolimus, tesirolimus, umirolimus, and zotarolimus.

In some embodiments, exemplary erectile dysfunction drugs suitable for use as an additional active ingredient include, but are not limited to Tadalafil, Vardenafil, Sildenafil, Alprostadil, Papaverine, and Phentolamine.

In some embodiments, the additional active ingredient is a non-steroidal anti-inflammatory drugs (NSAIDS). The NSAIDS can, for example, be selected from the following: choline salicylate (Arthropan) celecoxib (Celebrex); diclofenac potassium (Cataflam); diclofenac sodium (Voltaren, Voltaren XR); diclofenac sodium with misoprostol (Arthrotec); diflunisal (Dolobid); etodolac (Lodine, Lodine XL); fenoprofen calcium (Nalfon); flurbiprofen (Ansaid); ibuprofen (Advil, Motrin, Motrin IB, Nuprin); indomethacin (Indocin, Indocin SR); ketoprofen (Actron, Orudis, Orudis KT, Oruvail); magnesium salicylate (Arthritab, Bayer Select, Doan's Pills, Magan, Mobidin, Mobogesic); meclofenamate sodium (Meclomen); mefenamic acid (Ponstel); meloxicam (Mobic); nabumetone (Relafen); naproxen (Naprosyn, Naprelan); naproxen sodium (Aleve, Anaprox); oxaprozin (Daypro); piroxicam (Feldene); rofecoxib (Vioxx); salsalate (Amigesic, Anaflex 750, Disalcid, Marthritic, Mono-Gesic, Salflex, Salsitab); sodium salicylate (various generics); sulindac (Clinoril); tolmetin sodium (Tolectin); and valdecoxib (Bextra).

### 2. Powders

Another aspect of the present invention relates to powders prepared by the methods described above. In some embodiments, the dry powders of the present invention are tabletable powders. In some embodiments, the dry powders of the present invention can be tableted using standard tableting equipment producing more than 10,000 tablets per hour and the tablets can be prepared continuously for hours.

The present powder is prepared from the aqueous mixture by removing the water. The process for preparation of dry powder include various state of the art methods within pharmaceutical production like drying at increased temperature, vacuum drying, freeze drying, spray drying and spray granulation. Spray drying/spray granulation methods are the most preferred methods for preparation of tablettable powder.

In some embodiments, the powders of the present invention comprise an oil component and beta-cyclodextrin in a defined ratio which may preferably be expressed as a weight/weight (w/w) percentage of the oil component in the powder. In some embodiments, the powders of the present invention therefore comprise from 5% to 40%, 10% to 40%, 20% to 40%, 30% to 40%, 5% to 35%, 10% to 35%, 20% to 35%, 25% to 35%, 30% to 35%, at least 5%, at least 10%, at least 20%, at 30% or at least 35% w/w of an oil component, wherein w/w refers to the total weight of the oil component to the total weight of the powder.

In some embodiments, the oil component preferably comprises one or more omega-3 fatty acids or derivatives thereof. In some embodiments, the one or more omega-3 fatty acids or derivatives thereof are selected from omega-3 triglycerides, omega-3 ethyl esters, free omega-3 acids and/or pharmaceutically acceptable or food acceptable quality omega-3 fatty acid salts, alone or in combination. In some embodiments, one or more of these omega 3 fatty acids and derivatives thereof are the main omega-3 components used to prepare the dry powder. In some embodiments, the one or more omega-3 fatty acids or derivatives thereof are selected from omega-3 triglycerides, omega-3 ethyl esters, free omega-3 acids and/or pharmaceutically acceptable or food acceptable quality omega-3 fatty acid salts, alone or in combination. In some embodiments, one or more of these omega 3 fatty acids and derivatives thereof are the main omega-3 components in the dry powder. In some embodiments, one or more of these omega 3 fatty acids and derivatives thereof are the main oil components in the dry powder (e.g., the oil component in the powder comprises greater than about 70%, 80%, 90%, or 95% w/w of the specified omega-3 fatty acid or derivative thereof (omega-3 triglycerides, omega-3 ethyl esters, free omega-3 acids and/or pharmaceutically acceptable or food acceptable quality omega-3 fatty acid salts) wherein w/w refers to the total weight of the specified omega-3 fatty acid or derivative thereof per the total weight of the oil component). In some embodiments, one or more of these omega 3 fatty acids and derivatives thereof are from a marine source, such as fish, algae, or have been prepared from raw products from fish or algae. In some embodiments, one or more of these omega 3 fatty acids and derivatives thereof are from plants or vegetables or have been prepared from raw products from plants and vegetables. In some embodiments, the lipid component preferably comprises from about 30% to 60% w/w EPA and/or DHA. In some embodiments, the lipid component, and thus the powders, may comprise an additional active ingredient as described in detail above.

In some embodiments, the dry powder comprises omega-3 triglycerides. In some embodiments, the dry powder comprises omega-3 ethyl esters. In some embodiments, the dry powder comprises free omega-3 acids. In some embodiments, the dry powder comprises pharmaceutically acceptable or food acceptable quality of omega-3 fatty acid salts. In some embodiments, the omega-3 fatty acids or derivatives are preferably selected from EPA and DHA and combinations thereof. In some embodiments, the omega 3 fatty acids and derivatives thereof comprise more EPA than DHA (i.e., the ratio of EPA: DHA is greater than 1:1). In some embodiments, the omega 3 fatty acids and derivatives thereof comprise more DHA than EPA (i.e., the ratio of DHA: EPA is greater than 1:1). In some embodiments, the omega 3 fatty acids and derivatives thereof (e.g., triglycerides, ethyl esters, free acids or salts thereof, alone or in combination) comprise are enriched for EPA, e.g., more than 90% w/w of the total omega 3 fatty acids and derivatives thereof in the powder is EPA where the w/w is the weight of EPA per total weight of fatty acids in the powder. In some embodiments, the omega 3 fatty acids and derivatives thereof (e.g., triglycerides, ethyl esters, or free acids) are enriched for DHA, e.g., more than 90% w/w of the total omega 3 fatty acids and derivatives thereof in the powder is DHA where the w/w % is the weight of DHA per total weight of fatty acids in the powder.

In some embodiments, the powders may be further characterized according to their total omega-3 content. In some embodiments, the dry powders of the present invention comprise more than 5% w/w, more than 10% w/w, more than 15% w/w, more than 20% w/w, more than 25% w/w or more than 30% w/w of omega-3 triglycerides, omega-3 ethyl esters, free omega-3 acids and/or pharmaceutically acceptable or food acceptable quality of omega-3 fatty acid salts where w/w refers to the total weight of the omega-3 triglycerides, omega-3 ethyl esters, free omega-3 acids and/or pharmaceutically acceptable or food acceptable quality of omega-3 fatty acid salts per the total weight of the powder.

In some embodiments, the dry powders of the present invention comprise a surfactant (e.g., a diglycerides composition). In some embodiments, the w/w percent of the surfactant in the dry powder may be less than the w/w percent of surfactant used to prepare the powder, especially where the surfactant is water soluble. The surfactant or combination of surfactants is preferably included in the powder in a defined ratio as compared to the oil component.

Accordingly, in some embodiments, the dry powders of the present invention comprise a surfactant (e.g., a diglycerides composition) in a defined ratio to the amount of the oil component, preferably from 0.1% to 10% w/w, 0.1% to 20% w/w, 0.1% to 30% w/w, 1% to 10% w/w, 1% to 20% w/w, 1% to 30% w/w, 2% to 10% w/w, 2% to 20% w/w, 2% to 30% w/w, 5% to 10% w/w, 5% to 20% w/w, 5% to 30% w/w, 10% to 20% w/w, 10% to 30% w/w, 15% to 30% w/w, 18% to 30% w/w, 20% to 30% w/w, or 10% to 35% w/w surfactant or combination of surfactants, wherein w/w refers to the total weight of surfactant (or combination thereof) to the total weight of the oil component including the surfactant.

In some embodiments, the surfactant is a surfactant approved for use for preparation of pharmaceutical products and or approved for use in food products. In some embodiments, the surfactant is a surfactant approved for use for preparation of pharmaceutical products. In some embodiments, the surfactant is a surfactant approved for use for preparation of approved for use in food products. In some embodiments, the surfactant is a ionic surfactant; preferably a negatively charged surfactant. In some embodiments, the surfactant is a non-ionic surfactant. In some embodiments, the surfactant is a naturally occurring surfactant. In some embodiments, the surfactant is a surfactant produced synthetically or party produced synthetically. In some embodiments, the surfactant is a derivative of a fatty acid. In some embodiments, the surfactant is a derivative of glycerol. In some embodiments, the surfactant is selected among substances that are permitted to be used as food additives for use within the European Union, US or Asia. In some embodiments, the surfactant is selected among substances listed on the GRAS list. In some embodiments, the surfactant is selected among the following compounds: E 400 alginic acid, E401 sodium alginate, E402 potassium alginate, E403 ammonium alginate, E404 calcium alginate, E430 polyoxyethene (8) stearate, E431 polyoxyethene (40) stearate, E432 polyoxyethene (20) sorbitan monolaurate (polysorbate 20), E433 polyoxyethene (20) sorbitan monooleate (polysorbate 80), E434 polyoxyethene (20) sorbitan monopalmitate (polysorbate 40), E435 polyoxyethene (20) sorbitan monostearate (polysorbate 60), E436 polyoxyethene (20) sorbitan tristearate (polysorbate 65), E470a sodium, potassium and calcium salts of fatty acids, E470b magnesium salts of fatty acids, E471 mono- and diglycerides of fatty acids (glyceryl monostearate, glyceryl distearate) and other monoglycerides of fatty acids and diglycerides of fatty acids, E472 acetic acid esters of mono- and diglycerides of fatty acids, E472b lactic acid esters of mono- and diglycerides of fatty acids, E472c citric acid esters of mono- and diglycerides of fatty acids, E472d tartaric acid esters of mono- and diglycerides of fatty acids,E472e mono- and diacetyl tartaric acid esters of mono- and diglycerides of fatty acids, E472f mixed acetic and tartaric acid esters of mono- and diglycerides of fatty acids, E472g succinylated monoglycerides, E473 sucrose esters of fatty acids, E474 sucroglycerides, E475 polyglycerol esters of fatty acids, E476 polyglycerol polyricinoleate, E477 propane-1,2-diol esters of fatty acids, propylene glycol esters of fatty acids, E478 lactylated fatty acid esters of glycerol and propane-1,E479b thermally oxidized soya bean oil interacted with mono- and diglycerides of fatty acid, E480 dioctyl sodium sulphosuccinate, E481 sodium stearoyl-2-lactylate, E482 calcium stearoyl-2-lactylate, E483 stearyl tartrate, E484 stearyl citrate, E485 sodium stearoyl fumarate, E486 calcium stearoyl fumarate, E487 sodium laurylsulphate, E488 ethoxylated mono- and di-glycerides, E489 methyl glucoside-coconut oil ester, E490 propane-1,2-diol, E491 sorbitan monostearate, E492 sorbitan tristearate, E493 sorbitan monolaurate, E494 sorbitan monooleate, E495 sorbitan monopalmitate, E496 sorbitan trioleate, E497 polyoxypropylene-polyoxyethylene polymers and E498 partial polyglycerol esters of polycondensed fatty acids of castor oil. The term fatty acid includes any natural saturated fatty acids, monounsaturated fatty acids and polyunstaturated fatty acids and mixtures thereof.

In some embodiments, the surfactant or a surfactant mixture that has a HLB value of from 1 to 20. The HLB (Hydrophilic Lipophilic Balance) value for a given surfactant is measure of the degree to which the surfactant is hydrophilic or lipophilic. The figure is dependent on which functional groups thatare present in the surfactant molecule and where in the molecule these functional groups are located. Surfactants with HLB value of less than 10 are soluble in lipids, while surfactants with HLB values higher than 10 are soluble in water. The HLB values of the various surfactants are available from various commercial and scientific sources; see for example Surfactants Classified by HLB Numbers on sigmaaldrich.com or basic teaching books in pharmaceutical sciences like A.T. Florence and D. Attwood: Physicochemical Principles of Pharmacy, Pharmaceutical Press, 2004 on page 240. The HLB value for some preferred surfactants according to the present invention are: mono-and diglycerides (HLB=appr.2-5 (depending on ratio, the more diglyceride the lower HLB value)), sorbitan esters HLB values around 4-5 (sorbitan oleate HLB=4.3, sorbitan monostearate HLB=4.7, sorbitan stearate HLB=4.7) and polysorbates HLB values around 15.

In some preferred embodiments, the surfactant is selected among mono- and diglycerides of fatty acids, sorbitan esters with fatty acids and polysorbates or mixtures thereof. In some embodiments, the surfactant is not a phospholipid and most preferably is not a naturally occurring phospholipid. In some embodiments, the surfactant is not a triglyceride and most preferably is not a naturally occurring triglyceride. In some embodiments, the surfactant is not a free fatty acid and most preferably is not a naturally occurring free fatty acid. In some preferred embodiments, the surfactant is not a salt or ester of EPA, DHA or other long chain (greater than 20 carbons) omega-3 fatty acid. 3) Tablets

Another aspect of the present invention relates to tablets formed from the powders described above. As described above, in some embodiments, the dry powders of the present invention are tabletable powders. In some embodiments, the dry powders of the present invention can be tableted using standard tableting equipment producing more than 10,000 tablets per hour and the tablets can be prepared continuously for hours. In some preferred embodiments, the tablets have a crushing strength of greater than 3, 4, 5, 6, 7, 8, 9, 10, 11, 12, 13, 14 or 15 kN, or from about 3 to 50, 3 to 40, 3 to 30, 3 to 20, 5 to 50, 5 to 40, 5 to 30, 5 to 20, 5 to 10, 7 to 50, 7 to 40, 7 to 30, 7 to 20, 10 to 50, 10 to 40, 10 to 30, or 10 to 20 kN.

In some embodiments, the tablets of the present invention comprise an oil component and beta-cyclodextrin in a defined ratio which may preferably be expressed as a weight/weight (w/w) percentage of the oil component in the powder. In some embodiments, the tablets of the present invention therefore comprise from 5% to 40%, 10% to 40%, 20% to 40%, 30% to 40%, 5% to 35%, 10% to 35%, 20% to 35%, 25% to 35%, 30% to 35%, at least 5%, at least 10%, at least 20%, at 30% or at least 35% w/w of an oil component, wherein w/w refers to the total weight of the oil component to the total weight of the tablet.

In some embodiments, the oil component preferably comprises one or more omega-3 fatty acids or derivatives thereof. In some embodiments, the one or more omega-3 fatty acids or derivatives thereof are selected from omega-3 triglycerides, omega-3 ethyl esters, free omega-3 acids and/or pharmaceutically acceptable or food acceptable quality omega-3 fatty acid salts, alone or in combination. In some embodiments, one or more of these omega 3 fatty acids and derivatives thereof are the main omega-3 components used to prepare the dry powder. In some embodiments, the one or more omega-3 fatty acids or derivatives thereof are selected from omega-3 triglycerides, omega-3 ethyl esters, free omega-3 acids and/or pharmaceutically acceptable or food acceptable quality omega-3 fatty acid salts, alone or in combination. In some embodiments, one or more of these omega 3 fatty acids and derivatives thereof are the main omega-3 components in the dry powder. In some embodiments, one or more of these omega 3 fatty acids and derivatives thereof are the main oil components in the dry powder (e.g., the oil component in the powder comprises greater than about 70%, 80%, 90%, or 95% w/w of the specified omega-3 fatty acid or derivative thereof (omega-3 triglycerides, omega-3 ethyl esters, free omega-3 acids and/or pharmaceutically acceptable or food acceptable quality omega-3 fatty acid salts) wherein w/w refers to the total weight of the specified omega-3 fatty acid or derivative thereof per the total weight of the oil component). In some embodiments, one or more of these omega 3 fatty acids and derivatives thereof are from a marine source, such as fish, algae, or have been prepared from raw products from fish or algae. In some embodiments, one or more of these omega 3 fatty acids and derivatives thereof are from plants or vegetables or have been prepared from raw products from plants and vegetables. In some embodiments, the lipid component used to prepare the powder and thus the tablets may comprise an additional active ingredient as described in detail above.

In some embodiments, the tablet comprises omega-3 triglycerides. In some embodiments, the tablet comprises omega-3 ethyl esters. In some embodiments, the tablet comprises free omega-3 acids. In some embodiments, the tablet comprises pharmaceutically acceptable or food acceptable quality of omega-3 fatty acid salts. In some embodiments, the omega-3 fatty acids or derivatives are preferably selected from EPA and DHA and combinations thereof. In some embodiments, the omega 3 fatty acids and derivatives thereof comprise more EPA than DHA (i.e., the ratio of EPA: DHA is greater than 1:1). In some embodiments, the omega 3 fatty acids and derivatives thereof comprise more DHA than EPA (i.e., the ratio of DHA: EPA is greater than 1:1). In some embodiments, the omega 3 fatty acids and derivatives thereof (e.g., triglycerides, ethyl esters, free acids or salts thereof, alone or in combination) comprise are enriched for EPA, e.g., more than 90% w/w of the total omega 3 fatty acids and derivatives thereof in the powder is EPA where the w/w is the weight of EPA per total weight of fatty acids in the powder. In some embodiments, the omega 3 fatty acids and derivatives thereof (e.g., triglycerides, ethyl esters, or free acids) are enriched for DHA, e.g., more than 90% w/w of the total omega 3 fatty acids and derivatives thereof in the powder is DHA where the w/w % is the weight of DHA per total weight of fatty acids in the powder. In some embodiments, the lipid component preferably comprises from about 30% to 60% w/w EPA and/or DHA.

In some embodiments, the tablets may be further characterized according to their total omega-3 content. In some embodiments, the tablets of the present invention comprise more than 5% w/w, more than 10% w/w, more than 15% w/w, more than 20% w/w, more than 25% w/w or more than 30% w/w of omega-3 triglycerides, omega-3 ethyl esters, free omega-3 acids and/or pharmaceutically acceptable or food acceptable quality of omega-3 fatty acid salts where w/w refers to the total weight of the omega-3 triglycerides, omega-3 ethyl esters, free omega-3 acids and/or pharmaceutically acceptable or food acceptable quality of omega-3 fatty acid salts per the total weight of the tablet.

In some embodiments, the tablets of the present invention comprise a surfactant (e.g., a diglyceride composition). In some embodiments, the w/w percent of the surfactant (e.g., a diglyceride composition) in the tablet may be less than the w/w percent of surfactant used to prepare the powder used to make the tablet, especially where the surfactant is water soluble. The surfactant or combination of surfactants is preferably included in the powder in a defined ratio as compared to the oil component.

Accordingly, in some embodiments, the tablets of the present invention comprise a surfactant in a defined ratio to the amount of the oil component in the tablet, preferably from 0.1% to 10% w/w, 0.1% to 20% w/w, 0.1% to 30% w/w, 1% to 10% w/w, 1% to 20% w/w, 1% to 30% w/w, 2% to 10% w/w, 2% to 20% w/w, 2% to 30% w/w, 5% to 10% w/w, 5% to 20% w/w, 5% to 30% w/w, 10% to 20% w/w, 10% to 30% w/w, 15% to 30% w/w, 18% to 30% w/w, 20% to 30% w/w, or 10% to 35% w/w surfactant or combination of surfactants, wherein w/w refers to the total weight of surfactant (or combination thereof) to the total weight of the oil component including the surfactant.

In some embodiments, the surfactant is a surfactant approved for use for preparation of pharmaceutical products and or approved for use in food products. In some embodiments, the surfactant is a surfactant approved for use for preparation of pharmaceutical products. In some embodiments, the surfactant is a surfactant approved for use for preparation of approved for use in food products. In some embodiments, the surfactant is a ionic surfactant; preferably a negatively charged surfactant. In some embodiments, the surfactant is a non-ionic surfactant. In some embodiments, the surfactant is a naturally occurring surfactant. In some embodiments, the surfactant is a surfactant produced synthetically or party produced synthetically. In some embodiments, the surfactant is a derivative of a fatty acid. In some embodiments, the surfactant is a derivative of glycerol. In some embodiments, the surfactant is selected among substances that are permitted to be used as food additives for use within the European Union, US or Asia. In some embodiments, the surfactant is selected among substances listed on the GRAS list. In some embodiments, the surfactant is selected among the following compounds: E 400 alginic acid, E401 sodium alginate, E402 potassium alginate, E403 ammonium alginate, E404 calcium alginate, E430 polyoxyethene (8) stearate, E431 polyoxyethene (40) stearate, E432 polyoxyethene (20) sorbitan monolaurate (polysorbate 20), E433 polyoxyethene (20) sorbitan monooleate (polysorbate 80), E434 polyoxyethene (20) sorbitan monopalmitate (polysorbate 40), E435 polyoxyethene (20) sorbitan monostearate (polysorbate 60), E436 polyoxyethene (20) sorbitan tristearate (polysorbate 65), E470a sodium, potassium and calcium salts of fatty acids, E470b magnesium salts of fatty acids, E471 mono- and diglycerides of fatty acids (glyceryl monostearate, glyceryl distearate) and other monoglycerides of fatty acids and diglycerides of fatty acids, E472 acetic acid esters of mono- and diglycerides of fatty acids, E472b lactic acid esters of mono- and diglycerides of fatty acids, E472c citric acid esters of mono- and diglycerides of fatty acids, E472d tartaric acid esters of mono- and diglycerides of fatty acids, E472e mono- and diacetyl tartaric acid esters of mono- and diglycerides of fatty acids, E472f mixed acetic and tartaric acid esters of mono- and diglycerides of fatty acids, E472g succinylated monoglycerides, E473 sucrose esters of fatty acids, E474 sucroglycerides, E475 polyglycerol esters of fatty acids, E476 polyglycerol polyricinoleate—E477 propane-1,2-diol esters of fatty acids, propylene glycol esters of fatty acids, E478 lactylated fatty acid esters of glycerol and propane-1, E479b thermally oxidized soya bean oil interacted with mono- and diglycerides of fatty acid, E480 dioctyl sodium sulphosuccinate, E481 sodium stearoyl-2-lactylate, E482 calcium stearoyl-2-lactylate, E483 stearyl tartrate, E484 stearyl citrate, E485 sodium stearoyl fumarate, E486 calcium stearoyl fumarate, E487 sodium laurylsulphate, E488 ethoxylated mono- and di-glycerides, E489 methyl glucoside-coconut oil ester, E490 propane-1,2-diol, E491 sorbitan monostearate, E492 sorbitan tristearate, E493 sorbitan monolaurate, E494 sorbitan monooleate, E495 sorbitan monopalmitate, E496 sorbitan trioleate, E497 polyoxypropylene-polyoxyethylene polymers and E498 partial polyglycerol esters of polycondensed fatty acids of castor oil. The term fatty acid include any natural saturated fatty acids, monounsaturated fatty acids and polyunstaturated fatty acids and mixtures thereof.

In some embodiments, the surfactant or a surfactant mixture that has a HLB value of from 1 to 20. The HLB (Hydrophilic Lipophilic Balance) value for a given surfactant is measure of the degree to which the surfactant is hydrophilic or lipophilic. The figure is dependent on which functional groups that are present in the surfactant molecule and where in the molecule these functional groups are located. Surfactants with HLB value of less than 10 are soluble in lipids, while surfactants with HLB values higher than 10 are soluble in water. The HLB values of the various surfactants are available from various commercial and scientific sources; see for example Surfactants Classified by HLB Numbers on sigmaaldrich.com or basic teaching books in pharmaceutical sciences like A.T. Florence and D. Attwood: Physicochemical Principles of Pharmacy, Pharmaceutical Press, 2004 on page 240. The HLB value for some preferred surfactants according to the present invention are: mono-and diglycerides (HLB=appr.2-5 (depending on ratio, the more diglyceride the lower HLB value)), sorbitan esters HLB values around 4-5 (sorbitan oleate HLB=4.3, sorbitan monostearate HLB=4.7, sorbitan stearate HLB=4.7) and polysorbates HLB values around 15.

In some preferred embodiments, the surfactant is selected among mono- and diglycerides of fatty acids, sorbitan esters with fatty acids and polysorbates or mixtures thereof. In some embodiments, the surfactant is not a phospholipid and most preferably is not a naturally occurring phospholipid. In some embodiments, the surfactant is not a triglyceride and most preferably is not a naturally occurring triglyceride. In some embodiments, the surfactant is not a free fatty acid and most preferably is not a naturally occurring free fatty acid. In some preferred embodiments, the surfactant is not a salt or ester of EPA, DHA or other long chain (greater than 20 carbons) omega-3 fatty acid.The more preferred surfactants in tablets are diglycerides and the most preferred surfactants are diglycerides where one or more of the acids are omega-3 fatty acids. The typically most preferred diglycerides comprise of a mixture of diglyceride compounds where the fatty acid components in the mixture of diglyceride molecules can be saturated, monounsaturated and/or polyunsaturated, including omega-3 fatty acids like EPA and DHA.

In some embodiments, the tablets are coated. Suitable coatings include, but are not limited to, polyvinyl acetate, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, cellulose acetate trimellitate, and sodium alginate.

### EXAMPLES

### Example 1

### Preparation of 30:70 triglyceride:beta-cyclodextrin Complexes with diglycerides.

Beta-cyclodextrin (1000 g) was suspended in water at room temperature. A mixture of EPA and DHA (60% w/w) and various fatty acids as triglycerides, diglycerides and monoglycerides (430 g) was added. The mixture was stirred for 1 hour. The water was evaporated. The product was off-white, tabletable powder.

### Example 2

### Preparation of 30:70 triglyceride:beta-cyclodextrin Complexes Prepared with 7% diglycerides

The product was prepared as in example 1 with oil with glyceride composition (triglycerides/diglycerides/monoglycerides) as 91/7/1 area %. The product was a slurry with obvious oil layers, it was not possible to prepare dry powders from this mixture.

### Example 3

### Preparation of 30:70 triglyceride:beta-cyclodextrin Complexes Prepared with 19 area % diglycerides.

The product was prepared as in example 1 with oil with glyceride composition (triglycerides/diglycerides/monoglycerides) as 80/19/1 area %.

The product was dry, off-white to yellow powder. The powder was tabletable.

### Example 4

### Preparation of 30:70 triglyceride:beta-cyclodextrin Complexes Prepared with 24 area % diglycerides.

The product was prepared as in example 1 with oil with glyceride composition (triglycerides/diglycerides/monoglycerides) as 74/25/1 area %.

The product was dry, off-white powder. The powder was directly tabletable.

### Example 5

### Preparation of 30:70 triglyceride:beta-cyclodextrin complexes prepared with 32 area % diglycerides.

The product was prepared as in example 1 with oil with glyceride composition (triglycerides/diglycerides/monoglycerides) as 67/32/1 area %.

The product was dry, off-white powder. The powder was directly tabletable.

### Example 6

### Preparation of 30:70 triglyceride:beta-cyclodextrin complexes prepared with 34 area % diglycerides.

The product was prepared as in example 1 with oil with glyceride composition (triglycerides/diglycerides/monoglycerides) as 65/34/1 area %.

The product was dry, off-white powder. The powder was directly tabletable.

### Example 7

### Tablets prepared from 30:70 triglyceride:beta-cyclodextrin Complexes Prepared with 27 area % diglycerides.

Tablets comprising 96% (w/w) triglyceride:beta-cyclodextrin complexes prepared from oil with glyceride composition (triglycerides/diglycerides/monoglycerides) as 74/25/1 area % was prepared in a conventional tableting machine.

The tablets achieved a crushing strength of 9.1 kN.

### Example 8

### Preparation of triglyceride:beta-cyclodextrin Powder with Spray Granulation

The powder was prepared as in example 1, the method for water evaporation was spray granulation. The powder comprised of rounded particles with particle size distribution between 50-650 microns. The powder was directly tablettable.

### Example 9

### Preparation of 30:70 triglyceride:beta-cyclodextrin complexes with different content of diglyceride (DG) optionally added a surfactant.

The powder products were prepared as in example 1, spray granulated when complexes were achieved and tableted.

### Example 10

### Results for Powder and Tableting Experiments

The following Table provides results from experiments in which varying levels of surfactants, including diglycerides and other added surfactants, were used in powder formulations.

The first 10 experiments demonstrate that triglyceride omega-3 oil with a low content of diglycerides (8%) with added surfactants (1% or 10%) did not result in a powder for direct compaction (DC).

The next experiment shows that triglyceride omega-3 oil with relative high content of diglycerides (27%) result in powder for direct compaction. The next 3 experiments show that addition of surfactants to the composition results in a minor reduction of the crushing strength of the tablets, however, the powders were still tablettable.

The final 3 experiments have been performed with three different ethyl ester oils. EE60 comprise 60% omega-3, EE5325 comprise 33% EPA and 23%DHA and EE4020 comprise 40% EPA and 20% DHA. The results show that all ethyl ester oil composition tested, pain and with surfactants(s) formed powder that were directly compactable.

A control tablet experiment using a triglyceride omega-3 comprising 69% diglyceride showed lower crushing strength than comparative tablets comprising appr. 30% diglyceride (same oil loading in both tablets).

Tablets

| Oil | | | | | | | Crushing | |
|---|---|---|---|---|---|---|---|---|
| Raw | Oil | MG/DG/ | Surfactants | | | | strength | Friability |
| material | load | TG | Surfactant | HLB | Amount | Powder | (kN) | (%) |
| TG3322 | 30% | 1/8/91 | Span 85 | 1.8 | 1% | No | — | — |
| | | | Tween 40 | 15.6 | 1% | No | — | — |
| | | | Tween | 9.2 | 1% | No | — | — |
| | | | 40 + Span 85 | | | | | |
| | | | Span 20 | 8.6 | 1% | No | — | — |
| | | | Span 80 | 4.3 | 1% | No | — | — |
| | | | Tween 80 | 15 | 1% | No | — | — |
| | | | Tween 60 | 14.9 | 1% | No | — | — |
| | | | Span 85 | 1.8 | 10% | No | — | — |
| | | | Tween 40 | 15.6 | 10% | No | — | — |
| | | | Span 20 + | 12.1 | 10% | No | — | — |
| | | | Tween 40 | | | | | |
| TG3322 | 30% | 1/27/69 | None | — | — | DC | 9.1 | 99.9 |
| | | | | | | grade | | |
| | | | Span 85 | 1.8 | 1% | DC | 8.7 | 99.9 |
| | | | | | | grade | | |
| | | | Tween 40 | 15.6 | 0.1% | DC | 7.6 | 99.9 |
| | | | | | | grade | | |
| | | | Tween | 9.2 | 1% + 1% | DC | 7.6 | 99.9 |
| | | | 40 + Span | | | grade | | |
| | | | 85 | | | | | |
| EE60 | 30% | — | None | — | — | DC | 8.6 | 99.9 |
| | | | | | | grade | | |
| EE3525 | 30% | — | Tween 40 + | 9.2 | 1% + 1% | DC | 5.4 | 99.8 |
| | | | Span 85 | | | grade | | |
| EE4020 | 30% | — | Span 85 | 1.8 | 0.5% | DC | 6.9 | 99.9 |
| | | | | | | grade | | |

1. A composition comprising:
a dry powder comprising beta-cyclodextrin in an amount of from 60% to 90% w/w of said powder and a lipid component in an amount of from about 10% to 40% w/w of said powder, wherein said lipid component is characterized as having a surfactant content of from about 0.1% to 35% w/w of said lipid component.

2. The composition of claim 1, wherein said lipid component comprises an omega-3 fatty acid or derivative thereof selected from the group consisting of omega-3 triglycerides, omega-3 ethyl esters, omega-3 free fatty acids and salts of omega-3 fatty acids.

3. The composition of claim 1, wherein said omega-3 fatty acids or derivatives thereof have an EPA:DHA ratio of greater than 1:1.

4. The composition of claim 1, wherein said omega-3 fatty acids or derivatives thereof have a DHA:EPA ratio of greater than 1:1.

5. The composition of claim 2, wherein said omega-3 triglycerides are a marine oil.

6. The composition of claim 5, wherein said marine oil is selected from the group consisting of fish oil, squid oil and algal oil.

7. The composition of claim 2, wherein said omega-3 fatty acid or derivative thereof selected from the group consisting of omega-3 triglycerides, omega-3 ethyl esters, omega-3 free fatty acids and salts of omega-3 fatty acids comprises EPA and DHA fatty acids at a concentration of from 10% to 99% w/w of the fatty acids in said omega-3 triglycerides, omega-3 ethyl esters, omega-3 free fatty acids and salts of omega-3 fatty acids.

8. The composition of claim 2, wherein said omega-3 fatty acid or derivative thereof selected from the group consisting of omega-3 triglycerides, omega-3 ethyl esters, omega-3 free fatty acids and salts of omega-3 fatty acids comprises EPA and DHA fatty acids at a concentration of from 10% to 70% w/w of the fatty acids in said omega-3 triglycerides, omega-3 ethyl esters, omega-3 free fatty acids and salts of omega-3 fatty acids.

9. (canceled)

10. The composition of claim 1, wherein said surfactant is selected from the group consisting of mono- and diglycerides of fatty acids, sorbitan esters of fatty acids, and polysorbates and combinations thereof

11. The composition of claim 1, wherein said surfactant is selected from the group consisting of mono- and diglycerides of fatty acids and combinations thereof.

12. The composition of claim 1, wherein said surfactant is a diglyceride of fatty acids.

13. The composition of claim 12, wherein said diglycerides of fatty acids comprise a mixture of diglyceride compounds wherein the fatty acid components of the diglycerides compounds are selected from saturated, monounsaturated and polyunsaturated fatty acids.

14. The composition of claim 13, wherein said polyunsaturated fatty acids are omega-3 fatty acids.

15. The composition of claim 14, wherein said omega-3 fatty acids are selected from EPA and DHA.

16. The composition of claim 12, wherein the concentration of said surfactant in said lipid component is from 10% to 35% w/w of said lipid component.

17. The composition of claim 1, wherein said surfactant is not an added naturally occurring surfactant selected from the group consisting of naturally occurring phospholipids, triglycerides and free fatty acids or a salt or ester of a long chain omega-3 fatty acid.

18. The composition of claim 1, wherein said powder composition is spray granulated.

19. The composition of claim 17, wherein said powder composition is spray granulated and has a particle size distribution of 50-650 microns.

20. (canceled)

21. A tableted lipid formulation comprising beta-cyclodextrin in a concentration of from 60% to 90% w/w of said tablet and a lipid component in a concentration of from 10% to 40% w/w of said tablet, wherein said lipid component is characterized as having a surfactant or diglyceride content of from 0.1% to 35% w/w of said lipid component, wherein said tablet has a crushing strength of greater than 3 kN.

22-44. (canceled)

45. A process for making a tabletable lipid powder comprising:
combining an aqueous solution of beta-cyclodextrin with a lipid component in an amount of from 10% to 40% w/w of said beta-cyclodextrin said solution, wherein said lipid component comprises one or more surfactants at a concentration of from 0.1% to 35% w/w of said lipid component;
mixing said aqueous solution of beta-cyclodextrin and said lipid component to provide a mixture; and removing water from said mixture to provide a dry powder.

46-69. (canceled)
